(19) United States
(12) Patent Application Publication
(10) Pub. No.: US 2011/0038822 A1
(43) Pub. Date: Feb. 17, 2011

(54) ANTIPERSPIRANT EMULSION PRODUCTS AND PROCESSES FOR MAKING THE SAME

(75) Inventors: Brittany Phipps, Scottsdale, AZ (US); Thomas Doering, Scottsdale, AZ (US)

Correspondence Address: THE DIAL CORPORATION, 19001 N. Scottsdale Road, SCOTTSDALE, AZ, 85255, US

(73) Assignee: The Dial Corporation, Scottsdale, AZ (US)

(21) Appl. No.: 12/540,532

(22) Filed: Aug. 13, 2009

(51) Int. Cl. A61K 8/28; A61K 8/18; A61Q 15/00

(52) U.S. Cl. 424/66; 424/65

(57) ABSTRACT

Antiperspirant products and processes for forming antiperspirant products are provided. One antiperspirant product comprises a water-in-oil emulsion having a water phase and an oil phase. The water phase comprises an active antiperspirant compound and water. The oil phase comprises cetyl PEG/PPG-10/1 dimethicone and a hydrophobic carrier. One process comprises combining water and an active antiperspirant compound and heating the water and the active antiperspirant compound to form a water phase. A carrier, a high MW polyethylene, and cetyl PEG/PPG-10/1 dimethicone are combined, the polyethylene is melted and an oil phase is formed. The water phase and oil phase are mixed.

### FIELD OF THE INVENTION

The present invention generally relates to antiperspirant products and processes for making them, and more particularly relates to antiperspirant emulsion products that exhibit antiperspirant efficacy with the enhanced feel of a deodorant and processes for making them.

### BACKGROUND OF THE INVENTION

Antiperspirants and deodorants are popular personal care products used to prevent or eliminate sweat and body odor caused by sweat. Antiperspirants typically prevent the secretion of sweat by blocking or plugging sweat-secreting glands, such as those located at the underarms. Deodorants counteract or mask the unwanted odors caused by bacterial flora in secreted sweat.

Antiperspirant sticks are desired by a large majority of the population because of the presence of antiperspirant active compounds that block or prevent the secretion of sweat and the accompanying odors thereof and because of their ease of application. The antiperspirant product is applied to the skin by swiping or rubbing the stick across the skin, typically of the underarm. However, antiperspirant users often are disappointed in the chalky, brittle, and/or crumbly application of the stick across the skin. Deodorants, on the other hand, typically provide a better “glide” of the deodorant product across the underarm skin. The term “glide” typically is used to denote the friction between the antiperspirant and/or deodorant product and the skin. The smoother the glide, or the less friction between the product and the skin, the more desirable the product to users. While deodorants typically exhibit smoother glide than antiperspirant sticks, they do not prevent or eliminate the secretion of sweat as do antiperspirants because they do not contain active antiperspirant compounds. Active antiperspirant compounds generally cannot be added to deodorants because the alkalinity of the deodorants cause the antiperspirant compounds, typically acidic, to precipitate or settle out of deodorants. Thus, there is a need for antiperspirant products that exhibit antiperspirant efficacy with the feel of deodorants.

In addition to active antiperspirant compounds, antiperspirant sticks generally contain structurants. The structurants are used to give a solid structure or consistency to the stick. The high melting points of certain structurants, however, are undesirable during manufacturing because they can lead to inconsistent or unpredictable yields of the antiperspirant products and because they can increase the costs of manufacturing. Thus, there is a need for antiperspirant products that can be manufactured at relatively lower temperatures.

Accordingly, it is desirable to provide antiperspirant products that exhibit antiperspirant efficacy with the feel of deodorants. It is also desirable to provide antiperspirant products that can be manufactured at relatively lower temperatures than conventional antiperspirants. In addition, it is desirable to provide processes for making such antiperspirant products. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

In accordance with an exemplary embodiment, an antiperspirant product is provided. The antiperspirant product comprises a water-in-oil emulsion having a water phase and an oil phase, the water phase comprising an active antiperspirant compound and water and the oil phase comprising cetyl PEG/PPG-10/1 dimethicone and a hydrophobic carrier.

In accordance with another exemplary embodiment, an antiperspirant product is provided. The antiperspirant product comprises a water-in-oil emulsion having a water phase and an oil phase, the water phase comprising an active antiperspirant compound and water and the oil phase comprising an emulsifier, a high molecular weight polyethylene, and a low molecular weight synthetic wax, the low molecular weight synthetic wax present in an amount sufficient to lower the melting point of the high molecular weight polyethylene.

In accordance with an exemplary embodiment, a process for making an antiperspirant product is provided. The process comprises the steps of combining water and an active antiperspirant compound and heating the water and the active antiperspirant compound to form a water phase. A carrier, high molecular weight polyethylene, and cetyl PEG/PPG-10/1 dimethicone are combined. The polyethylene is melted and an oil phase is formed. The water phase and oil phase are mixed.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The various embodiments contemplated herein relate to antiperspirant emulsion products with antiperspirant efficacy that exhibit improved skin feel. The various embodiments of the antiperspirant emulsion products comprise cetyl PEG/PPG-10/1 dimethicone as an emulsifier. It has been unexpectedly found that the use of cetyl PEG/PPG-10/1 dimethicone as an emulsifier in an antiperspirant product causes the antiperspirant product to exhibit skin feel characteristics that are typical of deodorant products. For example, with cetyl PEG/PPG-10/1 dimethicone, the antiperspirant emulsion products glide onto skin with less friction, that is, in a smoother manner, than conventional antiperspirants while still maintaining a solid consistency for easy application. The various embodiments of the antiperspirant emulsion products also comprise water and volatile carrier fluids that provide a cooling effect to the skin when they evaporate upon application to the skin. Further, in various embodiments, the antiperspirant emulsion products comprise a unique combination of structurants that result in less white residue on the skin and that cause the antiperspirant emulsion products to be manufacturable at lower temperatures.

In this regard, in one exemplary embodiment of the present invention, the antiperspirant emulsion product, hereinafter referred to as the antiperspirant product, is a water-in-oil emulsion comprising a water phase mixed with an oil phase. Preferably, the antiperspirant product comprises a water phase in an amount of about 35 to about 45 weight percent (wt. %) of the total antiperspirant product and an oil phase in an amount of about 55 to about 65 wt. % of the total antiperspirant product. The antiperspirant product preferably has a soft, non-flowing, solid composition that can be rubbed or wiped across the skin, particularly the underarm. However, the various embodiments are not so limited and the antiperspirant product can also have a gel, cream, or lotion consistency. The solid composition is substantially snow white in color, thus suggesting a clean and/or sterile nature.

The water phase of the antiperspirant product comprises a water-soluble active antiperspirant compound. Active antiperspirant compounds contain at least one active ingredient, typically metal salts, that are thought to reduce sweating by diffusing through the sweat ducts of apocrine glands (sweat glands responsible for body odor) and hydrolyzing in the sweat ducts, where they combine with proteins to form an amorphous metal hydroxide agglomerate, plugging the sweat ducts so sweat can not diffuse to the skin surface. Some active antiperspirant compounds that may be used in the antiperspirant product include astringent metallic salts, especially inorganic and organic salts of aluminum, zirconium, and zinc, as well as mixtures thereof. Particularly preferred are aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₐCl_{b} x (H₂O), wherein a is from 2 to about 5; a and b total to about 6; x is from 1 to about 6; and wherein a, b, and x may have non-integer values. Exemplary zirconium salts include those having the general formula ZrO(OH)₂₋ₐClₐ x (H₂O), wherein a is from about 1.5 to about 1.87, x is from about 1 to about 7, and wherein a and x may both have non-integer values. Particularly preferred zirconium salts are those complexes that additionally contain aluminum and glycine, commonly known as ZAG complexes. These ZAG complexes contain aluminum chlorohydroxide and zironyl hyroxy chloride conforming to the above-described formulas. Examples of active antiperspirant compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof. In a preferred embodiment, the antiperspirant compound is aluminum zirconium tetrachlorohydrex glycine. In a more preferred embodiment, the antiperspirant compound comprises aluminum zirconium tetrachlorohydrex glycine at an active level of about 18 to about 24 wt. % of the total antiperspirant product.

In an exemplary embodiment, the water phase also comprises at least one water soluble carrier/solubilizer present in a sufficient amount to solubilize or disperse the water phase ingredients of the antiperspirant product. Such carriers/solubilizers suitable for use in the antiperspirant product include, but are not limited to, propylene glycol, glycerol, dipropyl glycol, ethylene glycol, butylene glycol, propylene carbonate, dimethyl isosorbide, hexylene glycol, ethanol, n-butyl alcohol, n-propyl alcohol, isopropyl alcohol, and the like. In a preferred embodiment, the water phase comprises propylene glycol and, in a more preferred embodiment, the water phase comprises propylene glycol in an amount of about 8 to about 12 wt. % of the total antiperspirant product. In addition to the carrier/solubilizer, the antiperspirant product comprises water. As noted above, the water evaporates from the antiperspirant product upon application of the antiperspirant product to the skin, providing a cooling sensation to the skin.

The water phase also may comprise optional ingredients that serve a particular purpose. In one exemplary embodiment, the water phase comprises an activator for the active antiperspirant compound. In a preferred embodiment, the water phase comprises calcium chloride and in a more preferred embodiment comprises calcium chloride in an amount of about 0.7 to about 2 wt. % of the total antiperspirant product.

The oil phase of the antiperspirant product comprises an emulsifier of cetyl PEG/PPG-10/1 dimethicone, in accordance with an exemplary embodiment. Cetyl PEG/PPG-10/1 dimethicone is a copolymer of cetyl dimethicone and an alkoxylated derivative of dimethicone containing an average of 10 moles of ethylene oxide and 1 mole of propylene oxide. As noted above, the use of cetyl PEG/PPG-10/1 dimethicone as an emulsifier in the antiperspirant product causes the antiperspirant product to exhibit skin feel characteristics that are typical of deodorant products. For example, with cetyl PEG/PPG-10/1 dimethicone, the antiperspirant products glide onto the skin with less friction while still maintaining a solid consistency for easy application. In addition, when applied, the antiperspirant products are moister than typical antiperspirants and thus give the skin a moister and softer feel. In a preferred embodiment, the oil phase comprises cetyl PEG/PPG-10/1 dimethicone in an amount of about 1 to about 4 wt. % of the total antiperspirant product.

Further included in the oil phase of the antiperspirant product is at least one structurant and/or gellant (hereinafter referred collectively as structurant) that facilitates the solid consistency of the antiperspirant stick product. Naturally-occurring or synthetic waxy materials or combinations thereof can be used as such structurants. Examples of these waxy materials include those fatty alcohols that are solid at room temperature and hydrocarbon waxes or silicone waxes. Such materials are widely available, and by suitable selection of the materials themselves and their concentrations in the formulation, it is possible to obtain either a soft solid or a firm solid. In a preferred embodiment, the oil phase comprises a high molecular weight (MW) polyethylene. As used herein, the term “high molecular weight polyethylene” or “high MW polyethylene” means polyethylene having a molecular weight of 200 to 5000 daltons (Da). In a more preferred embodiment, the oil phase comprises high MW polyethylene having a molecular weight of about 500 Da. In another preferred embodiment, the oil phase comprises high MW polyethylene in an amount of about 5 to about 10 wt. % of the total antiperspirant product. In this regard, polyethylene can be used in smaller amounts as a structurant in the antiperspirant products than other structurants, such as stearyl alcohol, that can exhibit undesirable properties in the antiperspirant product. Stearyl alcohol is commonly used as a structurant in solid stick underarm products. However, stearyl alcohol has a tendency to leave visible white deposits on the skin, and the deposits can also transfer onto clothing when the clothing comes into contact with the skin. Accordingly, in another preferred embodiment, the oil phase comprises substantially no stearyl alcohol. The term “substantially no stearyl alcohol” as used herein means no stearyl alcohol or stearyl alcohol in an amount that is sufficiently small so that it would not cause visible white residue to deposit on skin and/or clothing after application of the antiperspirant product to the skin.

In accordance with another exemplary embodiment, when high MW polyethylene is used in the oil phase as a structurant, the oil phase also comprises at least one low MW synthetic wax. In addition to facilitating the high MW polyethylene by serving a structurant function, the low MW synthetic wax also improves the manufacturing processes of the antiperspirant products. Generally, polyethylene has a relatively high melting point (70-100° C.) and, thus, as described in more detail below, the oil phase of the antiperspirant product must be heated to this high melting point to melt the polyethylene. However, this high temperature heating may result in higher manufacturing costs and unpredictable and/or non-repeatable yields of the antiperspirant product. The presence of an effective amount of low MW synthetic wax (synthetic wax having a molecular weight in the range of 1200-2900 Da) modifies the high MW polyethylene, lowering the melting point of the polyethylene. Accordingly, the presence of the low MW synthetic wax may result in lower manufacturing costs and higher yield of the antiperspirant product. In an exemplary embodiment, the low MW synthetic wax is present in the oil phase in an amount of about 0 to about 3 wt. % of the total antiperspirant product. In another exemplary embodiment, the low MW synthetic wax has a molecular weight of about 1800. In addition to improving hardness of the antiperspirant stick product, the low MW synthetic wax reduces syneresis and tackiness and also has a high refractive index (R.I.) that minimizes and/or prevents a white residue on the skin by masking the antiperspirant metallic salt(s) that stays upon the skin upon evaporation of the carrier(s), described in more detail below. As used herein, the term “high refractive index” means an refractive index no less than about 1.4. While the use of low MW synthetic wax to lower the melting point of high MW polyethylene is described herein in the context of an antiperspirant product comprising cetyl PEG/PPG-10/1 dimethicone as an emulsifier, it will be appreciated that low MW synthetic wax can be used to lower the melting point of high MW polyethylene in an antiperspirant emulsion product containing any suitable emulsifier.

The oil phase further comprises at least one hydrophobic carrier. An example of suitable hydrophobic carriers includes liquid siloxanes and particularly volatile polyorganosiloxanes, that is, liquid materials having a measurable vapor pressure at ambient conditions. The polyorganosiloxanes can be linear or cyclic or mixtures thereof. Preferred siloxanes include cyclomethicones, such as cyclotetramethicone, cyclopentamethicone, and cyclohexamethicone, and mixtures thereof. The carrier also may comprise, additionally or alternatively, nonvolatile silicones such as dimethicone and dimethicone copolyols. Examples of suitable dimethicone and dimethicone copolyols include polyalkyl siloxanes, polyalkylaryl siloxanes, and polyether siloxane copolymers.

The oil phase may also comprise a high R.I. hydrophobic compound. The high R.I. hydrophobic compound minimizes and/or prevents a white residue on the skin by masking the antiperspirant metallic salt that stays upon the skin upon evaporation of the carrier(s). Examples of high R.I. hydrophobic compounds for use in the antiperspirant products include C₁₂-C₁₅ alkyl benzoate, such as Finsolv TN® available from Innospec of the United Kingdom, PPG-14 butyl ether, and phenyl dimethicone. In a preferred embodiment, the oil phase comprises C₁₂-C₁₅ alkyl benzoate and, in a more preferred embodiment, the oil phase comprises C₁₂-C₁₅ alkyl benzoate in an amount of about 2 to about 12 wt. % of the total antiperspirant product.

In addition to the ingredients identified above, the antiperspirant product may comprise additives, such as those used in conventional antiperspirants. These additives include, but are not limited to, fragrances, including encapsulated fragrances, dyes, pigments, preservatives, antioxidants, moisturizers, and the like. These optional ingredients can be included in the antiperspirant product in an amount of 0 to about 20 wt. %.

The antiperspirant product according to various embodiments can be prepared by combining the ingredients of the water phase using sufficient agitation to prepare a mixture and then heating the water phase. Similarly, the ingredients of the oil phase are combined and the oil phase is heated to a temperature at least sufficient to melt the structurant. The oil phase and the water phase are mixed together to form a water-in-oil emulsion. Any additive, such as fragrance, is then added to the water-in-oil emulsion. The resulting liquid antiperspirant product is poured into suitable molds and then cooled to room temperature.

The following is an exemplary embodiment of an antiperspirant product, with each of the components set forth in weight percent of the antiperspirant product. The example is provided for illustration purposes only and is not meant to limit the various embodiments of the antiperspirant product in any way.

### EXAMPLE

| Water Phase | | Oil Phase | |
|---|---|---|---|
| Component | wt. % | Component | wt. % |
| Aluminum Zirconium | 18-24 | C12-C15 Alkyl Benzoate | 2-12 |
| Tetrachlorohydrex | | Polyethylene (MW = 500) | 5-10 |
| GLY | | | |
| Propylene glycol | 8-12 | Cetyl PEG/PPG-10/ | 1-4 |
| Calcium Chloride | 0.7-2 | 1 dimethicone | |
| Water | q.s. | Synthetic wax (MW = 1800) | 0-3 |
| | | Cyclomethicone | q.s. |
| Total Water Phase | 35-45 | Total Oil Phase | 55-65 |

| | |
|---|---|
| Fragrance | 0-2.5 |
| Total | 100 |

The antiperspirant product of the Example was prepared by combining the water and the calcium chloride in a mixing vessel and agitating the mixture until dissolution is achieved. The propylene glycol was added to this solution. Using sufficient agitation to maintain a vortex, the aluminum zirconium tetrachlorohydrex GLY was added to the solution and the solution was heated to about 80° C. Separately, all ingredients of the oil phase were combined and the oil phase mixture was heated to a temperature in the range of about 90-100° C. until the high MW polyethylene was melted. (The temperature range for melting the polyethylene may vary depending on the amount of low MW synthetic wax present in the oil phase.) The oil phase was cooled to about 80° C., the temperature was then maintained, and sufficient agitation to maintain a vortex was continued. Next, the water phase was slowly added to the oil phase by droplet at constant temperature, keeping both phases subject to agitation and between 75-80° C., to form a continuous emulsion. The speed of the agitation then was increased. For a 400 g batch of emulsion, the speed was 600 revolutions per minute (RPM). Once the water phase was fully added to the oil phase, rapid mixing and temperature were maintained for 10 minutes. The emulsion was cooled to 75° C., the fragrance was added, and the resulting antiperspirant product was poured into a mold and allowed to cool to room temperature.

Accordingly, antiperspirant products with antiperspirant efficacy that exhibit improved skin feel and processes for making such antiperspirant products have been provided. The various embodiments of the antiperspirant product comprise cetyl PEG/PPG-10/1 dimethicone as an emulsifier. The use of cetyl PEG/PPG-10/1 dimethicone as an emulsifier in an antiperspirant product causes the antiperspirant product to glide onto the skin with less friction, that is, in a smoother manner, than conventional antiperspirants while still maintaining a solid consistency for easy application. The various embodiments of the antiperspirant emulsion products also comprise water and volatile carrier fluids that provide a cooling effect to the skin when they evaporate upon application to the skin. Further, in various embodiments, the antiperspirant emulsion products comprise a unique combination of structurants that result in less white residue on the skin and that cause the antiperspirant emulsion products to be manufacturable at lower temperatures.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An antiperspirant product comprising a water-in-oil emulsion having a water phase and an oil phase, the water phase comprising an active antiperspirant compound and water and the oil phase comprising cetyl PEG/PPG-10/1 dimethicone and a hydrophobic carrier.

2. The antiperspirant product of claim 1, wherein the water phase is present in an amount of about 35 to about 45 wt. % of the antiperspirant product.

3. The antiperspirant product of claim 1, wherein the oil phase is present in an amount of about 55 to about 65 wt. % of the antiperspirant product.

4. The antiperspirant product of claim 1, wherein the water phase further comprises a soluble carrier/solubilizer.

5. The antiperspirant product of claim 1, wherein the water phase further comprises an activator for the active antiperspirant compound.

6. The antiperspirant product of claim 1, wherein the oil phase further comprises a structurant.

7. The antiperspirant product of claim 6, wherein the structurant comprises high MW polyethylene.

8. The antiperspirant product of claim 7, wherein the oil phase further comprises a low MW synthetic wax present in an amount sufficient to lower the melting point of the high MW polyethylene.

9. The antiperspirant product of claim 7, wherein the cetyl PEG/PPG-10/1 dimethicone is present in the oil phase in an amount of 2 to about 12 wt. % of the antiperspirant product.

10. The antiperspirant product of claim 1, wherein the antiperspirant product contains substantially no stearyl alcohol.

11. The antiperspirant product of claim 1, wherein the oil phase further comprises a high refractive index hydrophobic compound.

12. The antiperspirant product of claim 1, wherein the active antiperspirant compound comprises aluminum zirconium tetrachlorohydrex GLY, and the water phase further comprises propylene glycol and calcium chloride and wherein the hydrophobic carrier is a cyclomethicone and the oil phase further comprises C12-C15 alkyl benzoate, a high MW polyethylene, and a low MW synthetic wax.

13. An antiperspirant product comprising a water-in-oil emulsion having a water phase and an oil phase, the water phase comprising an active antiperspirant compound and water and the oil phase comprising an emulsifier, a high MW polyethylene, and a low MW synthetic wax, the low MW synthetic wax present in an amount sufficient to lower the melting point of the high MW polyethylene.

14. The antiperspirant product of claim 13, wherein the emulsifier is cetyl PEG/PPG-10/1 dimethicone.

15. The antiperspirant product of claim 13, wherein the water phase is present in an amount of about 35 to about 45 wt. % of the antiperspirant product.

16. The antiperspirant product of claim 13, wherein the oil phase is present in an amount of about 55 to about 65 wt. % of the antiperspirant product.

17. A process for making an antiperspirant product, the process comprising the steps of:
combining water and an active antiperspirant compound;
heating the water and the active antiperspirant compound to form a water phase;
combining a carrier, a high MW polyethylene, and cetyl PEG/PPG-10/1 dimethicone;
melting the high MW polyethylene and forming an oil phase; and
mixing the water phase and the oil phase.

18. The process of claim 17, wherein the step of mixing the water phase and the oil phase comprises mixing the water phase and the oil phase so that the water phase is present in an amount of about 35 to about 45 wt. % of the antiperspirant product and the oil phase is present in an amount of about 55 to 65 wt. % of the antiperspirant product.

19. The process of claim 17, wherein the step of combining a carrier, a high MW polyethylene, and cetyl PEG/PPG-10/1 dimethicone comprises combining the carrier, the high MW polyethylene, and cetyl PEG/PPG-10/1 dimethicone with a low MW synthetic wax present in an amount sufficient to lower the melting point of the high MW polyethylene.

20. The process of claim 17, further comprising, after the step of mixing the water phase and the oil phase, the step of adding an additive.
